(19) United States
(12) Patent Application Publication
(10) Pub. No.: US 2025/0381712 A1
(43) Pub. Date: Dec. 18, 2025

(54) Method for Preparing a Plurality of Condoms

(71) Applicant: Reckitt Benckiser Health Limited, Slough, Berkshire (GB)

(72) Inventors: Andrew Richard ARNOLD, Douglas (GB); Suchet KHAENYOOK, Bang Pakong, ChaCheongsao (TH); Manus SRIRING, Udonthani (TH)

(21) Appl. No.: 18/850,272

(22) PCT Filed: Mar. 31, 2023
(86) PCT No.: PCT/GB2023/050870

§ 371 Date: Sep. 24, 2024

(30) Foreign Application Priority Data
Mar. 31, 2022 (GB) 2204723.7

(51) Int. Cl. B29C 41/14; A61F 6/04; B29C 41/00; B29C 41/22; B29K 7/00; B29K 105/00; B29L 31/00; C08C 1/02

(52) U.S. Cl. CPC B29C 41/14; A61F 6/04; B29C 41/003; B29C 41/22; C08C 1/02; B29K 2007/00; B29K 2105/0044; B29K 2105/0064; B29L 2031/7538

(57) ABSTRACT

The present invention provides a method for preparing a plurality of condoms, comprising compounding one or more compositions comprising natural rubber latex to make a first batch of compounded latex, compounding one or more compositions comprising natural rubber latex to make a second batch of compounded latex, optionally, blending said first and second batches of compounded latex to make a compounded latex blend. A plurality of formers is dipped into the compounded latex blend to form a plurality of condoms, or a plurality of formers is dipped into the first batch of compounded latex to form a plurality of condoms and then said plurality of formers is dipped into the second batch of compounded latex to form a plurality of condoms. Each of the one or more compositions comprising natural rubber latex, which are used to make the first and second batch of compounded latex, has a zinc content less than 60 ppm. The invention further provides a plurality of condoms obtained or obtainable by this method.

The present invention relates to a method for preparing a plurality of condoms. More particularly, the present invention relates to a method for preparing a plurality of natural rubber latex condoms resulting in an improved proportion of condoms meeting quality testing requirements, therefore reducing wastage.

The ability of a condom to maintain its integrity throughout sexual activity is essential to its possible uses as a contraceptive and in preventing the spread of sexually transmitted infections. A condom must also be highly deformable, while at the same time being thin and flexible enough to allow sensitivity of touch and feel. Natural rubber latex (“NRL”) is a polymeric material that has been found to be suitable for this purpose and this is obtained by tapping rubber trees. NRL has been used as a raw material for manufacturing condoms for many years and at least 90% of the condoms currently on the market are NRL condoms. In particular, NRL may be used to prepare thin condoms. Thin condoms are desirable to some consumers, in part because they may afford less reduction in sensation and pleasure compared with condoms having thicker walls.

NRL typically comprises cis-1,4-polyisoprene together with small amounts of impurities, such as proteins, fatty acids, inorganic salts and the like. Rubber trees are tapped in order to obtain field natural rubber latex (FNRL). FNRL begins to decay just a few hours after it is tapped from a rubber tree, because bacteria can digest some of the non-rubber components and release volatile fatty acids, leading to putrefaction and destabilisation of the latex particles in the FNRL. The particle destabilisation also means that the latex begins to coagulate just a few hours after extraction. Therefore, in order to mitigate both of these issues, a preservative is typically added to the FNRL prior to concentration. Ammonia has traditionally been used as a preservative for FNRL as it functions not only as a biocide but also as a base to increase the pH, thereby effecting hydrolysis of the phospholipids and proteins present and reducing the number of branch points for coagulation to impart stability on the FNRL. However, there are some drawbacks associated with the use of ammonia as a preservative; for example, ammonia has a pungent smell. Alternative preservatives have therefore been investigated to allow the amount of ammonia preservative to be reduced. For example, a mixture of tetramethylthiuram disulfide and zinc oxide (TMTD/ZnO) may be used as a co-preservative with ammonia. Journal of Rubber Research (2021), 24:783-795 also suggests use of 1,2-benzisothiazolin-3-one (BIT) with ammonium laurate and a reduced ammonia content, amongst other possible preservatives.

In a typical condom manufacturing process, the preserved FNRL is then subjected to processing steps to form a concentrated natural rubber latex (CNRL), which is also referred to as a natural rubber latex concentrate. The FNRL is treated with diammonium hydrogen phosphate (DAP) to precipitate any magnesium ions present, because the presence of magnesium ions has been found to contribute to the instability and coagulation of the latex particles. The resulting latex is then subjected to a process of concentration to form a CNRL. The process of concentration is usually centrifugation; however, alternative concentration processes such as creaming are known. After this, the CNRL is stored before it is ready for use and preservation may also be required during storage in order to maintain the purity and stability of the CNRL. The CNRL is then used to manufacture dipped products, such as condoms.

When a batch of condoms is manufactured, quality control tests are carried out before rolling the condoms and sealing them in a package. For example, each condom in the batch is electrically tested for holes and any condoms failing this test are rejected. The percentage yield of condoms from each batch that pass the electrical tests is referred to as the electronic testing percentage yield. Other tests are carried out on condom samples from each batch. For example, a representative sample of a batch undergoes air inflation testing and the burst pressure (Bp) and burst volume (Bv) are measured and an acceptance quality level (AQL) is applied in accordance with international standards such as ISO 4074 which determines if the batch passes or fails. The requirements are particularly challenging for thin condoms, for example condoms having a thickness of less than 55 μm.

The inventors have found that condom quality can vary based on a number of factors affecting the CNRL, in particular thin condoms. These include natural factors affecting the FNRL, such the season of planting/harvesting, as well as the processing steps outlined above in order to form the CNRL. For example, the choice of preservative for preserving the FNRL and/or the CNRL, the amount of centrifugation and the storage time for the CNRL may affect the quality of the CNRL and in turn the quality of the condoms.

There remains a need to identify which properties of the FNRL and the CNRL have the greatest impact on condom quality. This means that a higher percentage of the condoms will pass electronic testing, leading to less wastage in the manufacturing process. This way, the specification of the FNRL and the CNRL can be refined in order to achieve the required condom quality in a higher yield for condoms formed from different batches.

According to a first aspect, the present invention provides a method for preparing a plurality of condoms, comprising:
(i) compounding one or more compositions comprising natural rubber latex to make a first batch of compounded latex;
(ii) compounding one or more compositions comprising natural rubber latex to make a second batch of compounded latex;
(iii) optionally, blending said first and second batches of compounded latex to make a compounded latex blend; and
(iv) dipping a plurality of formers into the compounded latex blend to form a plurality of condoms, or
(v) dipping a plurality of formers into the first batch of compounded latex to form a plurality of condoms and then dipping said plurality of formers into the second batch of compounded latex to form a plurality of condoms;
   wherein each of the one or more compositions comprising natural rubber latex, which are used to make the first and second batch of compounded latex, has a zinc content less than 60 ppm.

As detailed in the Examples, the inventors measured certain properties of 42 separate batches of CNRL obtained from the same supplier, prepared condoms using each batch and measured the electronic testing percentage yield of the condoms prepared from each batch. The inventors then carried out a bivariate correlation analysis between each of the properties of the CNRL batches and the electronic testing percentage yield. The inventors observed that there is a negative correlation between the gel content of the CNRL and the electronic testing percentage yield.

Without wishing to be bound by theory, gel formation can create inhomogeneity within the rubber latex. This in turn is thought to lead to non-uniform vulcanisation and create defects within the latex film (Nun-anan et al., Polym. Adv. Technol. 31, 2020, 44-59). However, it was not known what impact any such defects would have on dipped latex products, in particular condoms, for example whether the nature or extent of these defects would be sufficient to affect the electronic testing percentage yield.

As outlined above, the inventors have found that the gel content is in fact an important parameter to control in order to increase the electronic testing percentage yield. It is postulated that gel formation may result in a less elastic area of the film and generate weak spots at the interface between the gel and the remainder of the film. When the condom is partially stretched during electronic testing this weak interface may result in a micro-hole being generated that may be detected during testing.

The gel fraction in CNRL, which may be formed during storage of FNRL and/or CNRL, may be formed by at least one of the following types of bonding (Tarachiwin, L et al. Rubber Chem. technol. 2003, 76 (5), 1177-1184):
a) covalent, e.g. by C—C, C—O and C—S bonds between rubber chains;
b) ionic, e.g. by divalent or trivalent metal ions presenting in the latex;
c) hydrogen bonding e.g. between proteins presenting in the latex;

In Tarachiwin et al., it was observed that the addition of diammonium hydrogen phosphate (DAP) decreased the amount of ionic gel formation to a certain extent, because DAP removes divalent magnesium ions presenting in the latex. However, if an excess of DAP is added and the preservative used is TMTD/ZnO, it was observed that gel formation still took place even though most of the magnesium ions should have been removed from the latex. This suggests that divalent zinc ions from ZnO may also be involved in gel formation.

The zinc ions in the CNRL may promote gel formation by the formation of a zinc-amine complex (Riyajan S. et al., KGK. Jumi 2010) as shown below:

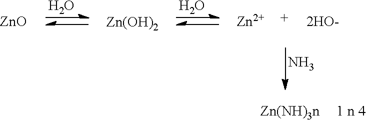

The inventors' hypothesis is supported by an experiment carried out by the inventors as detailed in Example 5. The inventors compared batches of CNRL with a high zinc content (465 ppm) and a low zinc content (32.4 ppm) and monitored the gel content of the CNRL batches during storage. After 84 days of storage, the inventors observed that the gel content of the batch with a zinc content of 465 ppm was significantly higher than the batch with zinc content of 32.4 ppm.

Therefore, in view of this finding, the inventors believed that by controlling the zinc content in the CNRL, this would decrease the gel formation in the CNRL, for example during storage of the CNRL. In turn, this would decrease the number of condoms that need to be discarded as a result of not meeting quality requirements and therefore reduce wastage.

According to a second aspect, the present invention provides a plurality of condoms obtained or obtainable by the method according to the first aspect.

The present invention will now be described further. In the following passages different aspects/embodiments of the invention are defined in more detail. Each aspect/embodiment so defined may be combined with any other aspect/embodiment or aspects/embodiments unless clearly indicated to the contrary. In particular, any feature indicated as being preferred or advantageous may be combined with any other feature or features indicated as being preferred or advantageous.

The present invention provides a method for preparing a plurality of condoms. Preferably, each condom of the plurality of condoms is a male condom, preferably one intended to cover substantially the entire penis. Alternatively, in some embodiments, each condom of the plurality of condoms is a female condom.

The first step of the method comprises compounding one or more compositions comprising natural rubber latex to make a first batch of compounded latex.

By “composition comprising natural rubber latex”, it is meant a concentrated natural rubber latex (CNRL) which is also referred to as a natural rubber latex concentrate. As described above, the field natural rubber latex (FNRL) is optionally preserved with a preservative, such as ammonia, before e.g. treatment with diammonium hydrogen phosphate (DAP, also referred to as DAHP) to precipitate out any magnesium ions present. However, trace amounts of magnesium ions may remain in the latex even after treatment with DAP. The resulting latex is then subjected to a process of concentration, resulting in an increase in the dry rubber content of the latex, to form a CNRL. The dry rubber content of CNRL may be at least about 60% by mass in accordance with ISO 2004:2010(E).

The process of concentration may be one or more centrifugation steps. The centrifugation process removes skim latex having smaller latex particles and approximately two thirds of the non-rubber components in the latex (S. Santipanusopon and S.-A. Riyajan, Physics Procedia 2 (2009), 127-134). However, components added to the FNRL, such as a preservative, may be retained even after centrifugation. Further preservative(s) may be added to the CNRL after the centrifugation process(es), which may be the same or different preservative(s) previously added to the FRNL.

One or more of the compositions comprising natural rubber latex may be stored before they are used for compounding, for example in a storage vessel at room temperature. One or more (preferably all) of the compositions comprising natural rubber latex may be stored for a storage period of: at least 7 days, at least 14 days, at least 21 days, at least 28 days, at least 30 days, at least 35 days, at least 42 days, at least 49 days, at least 56 days, at least 63 days, at least 70 days, at least 77 days, at least 84 days, at least 91 days, at least 98 days, at least 105 days, at least 112 days, at least 119 days, at least 126 days, at least 133 days or at least 140 days; and/or up to 147 days, up to 140 days, up to 133 days, up to 126 days, up to 119 days, up to 112 days, up to 105 days, up to 98 days, up to 91 days, up to 84 days, up to 77 days, up to 70 days, up to 63 days, up to 56 days, up to 49 days, or up to 42 days. In an embodiment, one or more (preferably all) of the compositions comprising natural rubber latex have been stored for a storage period of 7-147 days, preferably 14-98 days, preferably 21-70 days, preferably 28-56 days, before they are used for compounding. The storage period may be tailored to allow the CNRL to mature to achieve a particular mechanical stability time, for example at least 650 seconds.

In an embodiment, each of the one or more compositions comprising natural rubber latex is or has been stored for a storage period of at least 30 days before compounding, preferably 30-42 days. In another embodiment, each of the one or more compositions comprising natural rubber latex is stored for a storage period of at least 42 days before compounding.

As detailed in the Examples, the inventors observed that there is a positive correlation between the zeta potential of the CNRL and the electronic testing percentage yield. This suggests that an increase in latex stability would lead to an increase in the electronic testing percentage yield. In general, the inventors have observed that the stability of the natural rubber latex increases with storage time. This increase in stability is postulated to be due to the hydrolysis reaction between hydrolysable lipids (glycolipids and phospholipids) and any NH₄OH present e.g. from ammonia preservative, which leads to the formation of free fatty acids. The free fatty acids then adhere to the surface of the rubber particles, resulting in more negatively charged and well dispersed natural rubber particles (see, for example, Kumarn S. et al., Langmuir 2018, 34, 43, 12730-12738).

Further, a preservative and/or a stabiliser may also be added to the CNRL if it is stored prior to the compounding process. For example, ammonia may be used to preserve the CNRL and ammonium laurate may be used as a stabiliser.

As mentioned above, each of the one or more compositions comprising natural rubber latex has a zinc content of less than 60 ppm. By zinc content, this refers to zinc from zinc-containing ingredients in the composition; the source of the zinc may be ingredients that were previously added to the FNRL and/or the CNRL, such as a zinc-containing preservative, as well as any natural sources of zinc present in the latex.

The zinc content is measured immediately prior to compounding said one or more compositions or if any other compounding ingredients are added, immediately prior to mixing said one or more compositions with said compounding ingredients. The zinc content may be measured using the EDTA titrimetric method in accordance with ISO 2454:1995(E).

As explained above, by controlling the zinc content of each of the compositions comprising natural rubber latex, this reduces the gel formation during storage of FNRL and/or CNRL and in turn the gel content of CNRL immediately before the CNRL is used in compounding.

Preferably, each of the one or more compositions comprising natural rubber latex has a zinc content of less than 55 ppm, preferably less than 50 ppm, preferably less than 45 ppm, more preferably less than 40 ppm, still more preferably less than 35 ppm, less than 30 ppm, less than 25 ppm, or less than 20 ppm.

The zinc ions in the CNRL may originate from natural sources. For example, the soil may naturally contain some zinc ions or a fertiliser may be used which adds zinc to the soil. Reports of the natural zinc content of FNRL vary, and may not directly correlate with the content in the CNRL. Alternatively, sources of zinc ions may be introduced to the FNRL and/or the CNRL. For example, a zinc-containing preservative may be used to preserve the FNRL and/or the CNRL, such as ZnO/TMTD as detailed above.

In some embodiments, the zinc content is controlled by not adding any zinc-containing preservatives to the FNRL and/or the CNRL and reducing the zinc present from naturally occurring sources. Alternatively, in some embodiments, each of each the one or more compositions comprising natural rubber latex is preserved with a preservative comprising zinc before compounding, but the amount of this is carefully controlled. The preservative comprising zinc may be added to the FNRL and/or the CNRL. In an embodiment, the method comprises measuring the zinc content, and selecting the compositions with a zinc content of less than 60 ppm for compounding. In an alternative embodiment, the compositions with a zinc content greater than 60 ppm are modified in order to reduce the amount of zinc to less than 60 ppm. The zinc content can be reduced by known methods, whether chemical or physical. Preferably, the zinc content of each of the compositions comprising natural rubber latex used in the method of the invention is kept below 60 ppm (or below the preferred lower thresholds described above) throughout the preceding storage of the CNRL, and preferably also throughout the preceding storage of the FNRL.

Preferably, each of the one or more compositions comprising natural rubber latex has an ammonia content of from 0.65 to 1.5 wt %, more preferably from 0.65 to 1.0 wt % and most preferably 0.65 to 0.9 wt %. The ammonia content may be determined by acid-base titration with hydrochloric acid.

As discussed above, ammonia acts as a bactericide. Ammonia also helps to increase latex stability through hydrolysis of any phospholipids and glycolipids present in the latex to form free fatty acids, which adhere to the rubber particle surface. However, it is postulated that an excess of ammonia may contribute to gel formation with any zinc ions present.

As mentioned above, the one or more compositions comprising natural rubber latex are compounded to make a first batch of compounded latex. The one or more compositions may be mixed with other compounding ingredients prior to compounding. Suitable compounding processes and compounding ingredients are well-known in the art. Compounding ingredients include a vulcanising agent, an activator or accelerator, a stabilizer, an antioxidant and the like.

The second step of the method comprises compounding one or more compositions comprising natural rubber latex to make a second batch of compounded latex.

The definition of a “composition comprising natural rubber latex” is identical to said compositions used in the first step of the method. As for the first step of the method, each of the one or more compositions comprising natural rubber latex used in the second step of the method has a zinc content of less than 60 ppm.

Each of the optional or preferred features described above in relation to the one or more compositions comprising natural rubber latex as used in the first step of the method are equally applicable to the one or more compositions comprising natural rubber latex as used in the second step of the method. Further, the compounding step for the second step of the method is as described for the first step of the method, except that a different batch of composition or compositions comprising natural rubber latex are used in the second step.

In an embodiment, the method comprises compounding one or more compositions comprising natural rubber latex to make a third batch of compounded latex. In another embodiment, the method further comprises compounding one or more compositions comprising natural rubber latex to make a fourth batch of compounded latex. The definition of a “composition comprising natural rubber latex” is identical to said compositions used in the first step of the method. The number of batches of compounded latex used to prepare the plurality of condoms is not particularly limited and will be constrained by the capacity of the manufacturing facility.

In an embodiment, the compounding conditions (e.g. temperature, mixing time and the like) and compounding ingredients are the same for making all batches of compounded latex.

The third step of the method is an optional step and comprises blending said first and second batches of compounded latex to make a compounded latex blend. This allows multiple batches of compounded concentrated rubber latex to be blended together in order to prepare a single batch of condoms.

In embodiments where three or more batches of compounded latex are used to prepare the plurality of condoms, two or more of the compounded latex batches may be blended to make a compounded latex blend.

The fourth step of the method comprises dipping a plurality of formers into the compounded latex blend to form a plurality of condoms. The term “former” is known in the art and refers to a condom-shaped mould to which a polymeric coating composition is applied to form a condom. Formers can, for instance, be made from glass, plastic or ceramic.

In the fourth step of the method, the plurality of formers are dipped into the compounded latex blend to form a film on each of the formers. The term “film” refers to a thin layer of polymeric material, the thickness of the layer typically being on the order of several microns or tens of microns. Varying the speed of dipping and/or withdrawal can be used to control the thickness of the layer.

The fourth step preferably comprises dipping the plurality of formers into the compounded latex blend and drying the layer of the compounded latex blend to form a film on each of the formers. The drying step can be performed by evaporation in the open atmosphere or in an oven or evaporator. In some embodiments, the former is heated to facilitate drying.

In some embodiments, the layer of the compounded latex blend undergoes a chemical change during the drying step. For instance, if a cross-linking agent is present in the compounded latex blend, cross links may form during the drying step.

In embodiments where a drying step takes place to form a film on each of the formers, the formers may be dipped into the compounded latex blend for a second time to form a second film on each of the formers. In this case, preferably the second layer of the compounded latex blend is dried as for the first layer to form a second film on each of the formers. The drying conditions (e.g. temperature, time) for the second film may be the same compared to the drying step for the first film.

In some embodiments, three or more films of the compounded latex blend may be formed on each of the formers, by dipping the formers three or more times into the compounded latex blend and drying the formers as described above after each dipping step.

In an embodiment, after the plurality of formers were dipped into the compounded latex blend as described above, a bead may be formed at the opening of each condom e.g. by using brushes to roll the tops of the condoms to form a bead at the end of each condom whilst the formers rotate to prevent defective beads.

After the dipping step(s) and any drying and/or bead-forming steps, the film or films on the plurality of formers may be vulcanised e.g. by moving the formers into an oven.

The fourth step of the method comprises removing the plurality of formers to provide a plurality of condoms. In some embodiments, this involves stripping the film structure from each former. In some embodiments, the film structure is leached (e.g. in alkaline solution at a temperature of from 20 to 50° C.) from each former before the former is removed to provide the condom.

In an alternative embodiment, the fourth step of the method comprises dipping a plurality of formers into the first batch of compounded latex to form a plurality of condoms and then dipping said plurality of formers into the second batch of compounded latex to form a plurality of condoms.

This embodiment of the fourth step of the method is as described for the compounded latex blend, except that the first and second batches of compounded latex are not blended together before the dipping step.

Each of the optional or preferred features described above in relation to forming the plurality of condoms from the compounded latex blend are equally applicable to dipping a plurality of formers into the first batch of compounded latex to form a plurality of condoms and then dipping said plurality of formers into the second batch of compounded latex to form a plurality of condoms.

In one aspect of this embodiment, the plurality of formers are dipped into the first batch of compounded latex to form condoms which are removed from the formers and then said plurality of formers are dipped into the second batch of compounded latex to form a plurality of condoms. As described above for the compounded latex blend, the plurality of formers may be dipped two or more times into the first batch of compounded latex to form condoms with two or more film layers. The same applies to the second batch of compounded latex.

In embodiments where three or more batches of compounded latex are prepared, the formers are dipped into the first batch of compounded latex to form condoms which are removed from the formers, then said plurality of formers are dipped into the second batch of compounded latex to form condoms which are removed from the formers, then said plurality of formers are dipped into the third batch of compounded latex to form condoms which are removed from the formers and so on. Alternatively, the formers are dipped into the first batch of compounded latex to form condoms which are removed from the formers, then said plurality of formers are dipped into the second batch of compounded latex to form condoms which are removed from the formers, then said plurality of formers are dipped into a compounded latex blend made from third and fourth batches of compounded latex to form condoms which are removed from the formers and so on. Again, the plurality of formers may be dipped two or more times into any one of the batches of compounded latex and/or any one of the compounded latex blends to form condoms with multiple film layers.

Where more than one film layer is formed on the plurality of formers, a drying step takes place after the formation of the first film and before forming the second film and any subsequent films as outlined above. Preferably, a drying step takes place after the formation of the final film on the formers.

In an embodiment, each of the plurality of condoms has an average thickness of: up to 80 μm, up to 75 μm, up to 70 μm, up to 65 μm, up to 60 μm, up to 55 μm, up to 52 μm, or up to 50 μm; and/or at least 20 μm, at least 25 μm, at least 30 μm, at least 35 μm, at least 40 μm, at least 45 μm, or at least 50 μm. By “thickness” it is meant the wall thickness (i.e. the combined thickness of the layers as defined herein that form each condom), not including any bead formed at the opening of the condom.

In embodiments of the invention, each of the plurality of condoms has an average thickness of from 20 to 80 μm, 35 to 70 μm, or 55 to 70 μm. In an embodiment, the condom has a thickness of from 35 to 55 μm, or from 40 to 52 μm, or from 40 to 50 μm. By controlling the zinc content of the CNRL as outlined above, the present invention can be used to make thin condoms and achieve a higher ET % yield compared to using the same method but without the feature of each composition comprising natural rubber latex has a zinc content of less than 60 ppm.

The condoms of the invention may appear less yellow and/or have a lower protein content than those made from CNRL with higher zinc content.

In some embodiments, the method further comprises coating one or more surfaces of at least one condom of the plurality of condoms with a finishing powder. The one or more surfaces may be an inner and/or an outer surface, preferably an inner surface and an outer surface. “Inner” in the context of a male condom refers to the penis-facing side, whereas “outer” refers to the side facing the user's partner. In embodiments in which the method further comprises coating one or more surfaces of each of the condoms with a finishing powder, this is done prior to electronic testing, rolling, and before any lubricant is applied. The step of coating one or more surfaces of each condom with a finishing powder may comprise applying the finishing powder to the one or more surfaces as a powder or as a liquid slurry (preferably an aqueous slurry). In the latter embodiment, the water is allowed to evaporate to form the coating of the finishing powder on the one or more surfaces of the condom. Finishing powders are known in the art. They are typically alkaline, and are typically based on compounds such silica, talc, carbonates, corn starch and the like. They are used to prevent the surfaces of the condom from sticking to each other, and to assist with donning. In particular, including a finishing powder on an inner surface serves to prevent the condom from sticking to itself, while including a finishing powder on an outer surface also serves to prevent the condom from sticking to other condoms during production.

In some embodiments, the method further comprises electronic testing of each of the plurality of condoms for holes and all those condoms which do not pass the electronic testing are discarded. This may also be referred to as electrical testing.

Electronic testing may be carried out as follows. A condom is loaded carefully onto a metal or other electrically conductive mandrel and the condom is exposed to an electrical field. Rubber is not a conductor of electricity, so if the mandrel is affected by the electrical field then this indicates that there is a hole within the condom. A voltage in the range of 1200 to 2500 V is applied e.g. 1800 V±100 V. The starting voltage will depend on the nature of the condoms being tested e.g. the thickness of the condoms, and is chosen according to conventional practice. The voltage is gradually decreased until any holes in the latex film are detected. A threshold voltage is defined e.g. 1000 V, meaning that condoms fail the test if holes are detected at a voltage greater than the threshold voltage and pass the test if holes are only detected at a voltage less than the threshold voltage. Once all of the plurality of condoms have been tested, the % ET yield of each condom batch can be calculated using the following equation:

% ET yield=[Number of accepted condoms/Total number of condoms]×100

Preferably, one or more surfaces of each of the plurality of condoms has been coated with a finishing powder as outlined above, prior to the electronic testing.

In an embodiment, at least 90% of the condoms of the plurality of condoms obtained by the method pass the electronic testing and all those condoms which do not pass the electronic testing are discarded. In an alternative embodiment, at least 91% or 92% or 93%, or 94% or 95% or 96% of the condoms of the plurality of condoms obtained by the method pass the electronic testing and all those condoms which do not pass the electronic testing are discarded. For example, for every 100 condoms tested, on average only 10 condoms are discarded. This has a sustainability benefit as natural rubber latex condoms take a long time to biodegrade in the environment and so reducing wastage has environmental as well as financial benefits for the manufacturer.

In some embodiments, the method further comprises applying a dose of a lubricant to one or more surfaces of at least one of the condoms of the plurality of condoms to form a lubricated condom. The one or more surfaces may be an inner surface and/or an outer surface of the condom. Preferably, a dose of a lubricant is applied to at least the outer surface of the condom. Suitable lubricants for condoms are known in the art and are typically water-based or silicone oil-based. In some embodiments, the condom is rolled prior to the application of the lubricant. In this embodiment, the dose of lubricant may be applied at or near a tip of the rolled condom. The lubricant may then migrate along the rolls of the condom over time (including after the condom is sealed within a package, as discussed below). In this embodiment, the condom may already be within the package, or on a material which will form a part of the package (such as a foil package), at the time the dose of lubricant is applied. In embodiments in which the condom is sealed within a package (as described below), only the sealing step must necessarily take place after the lubricant is applied.

Alternatively, a dose of a lubricant may be applied to the condom prior to any rolling step. The lubricant may be applied in a variety of known ways, for example, by spraying, rolling over a sponge soaked with the lubricant, or the dose of lubricant may be applied to one or more spots along a length of the condom prior to rolling. It will be appreciated that if the lubricant is pre-applied to the condom in all or substantially all of the area where it is required, such that it does not need to migrate to the desired area, a higher viscosity of the lubricant may be tolerated.

The method may comprise further steps, such as rolling one or more of the plurality of condoms and/or sealing at least one of the plurality of condoms within a package. Suitable sealed packages for condoms are known in the art and may include, for instance, two sheets of a laminate material sealed along their edges around the condom. The laminate material may, for instance, include a layer of aluminium. Another possible sealed package is a plastic pot sealed with a film lid, or so-called “butter dish”. The condom is preferably provided within the package in a rolled state.

According to a second aspect, the present invention provides a plurality of condoms obtained or obtainable by the method according to the first aspect.

In some embodiments at least one of the condoms of the plurality of condoms further comprises, on one or more surfaces thereof, a finishing powder. The one or more surfaces may be an inner and/or an outer surface, preferably an inner surface and an outer surface. Suitable finishing powders and means for applying them are disclosed in relation to the first aspect.

In some embodiments at least one of the condoms of the plurality of condoms further comprises, on one or more surfaces thereof, a lubricant. The one or more surfaces may be an inner surface and/or an outer surface of the condom, preferably at least the outer surface. Suitable lubricants are described in relation to the first aspect.

The present invention will now be described in relation to the following non-limiting figures.

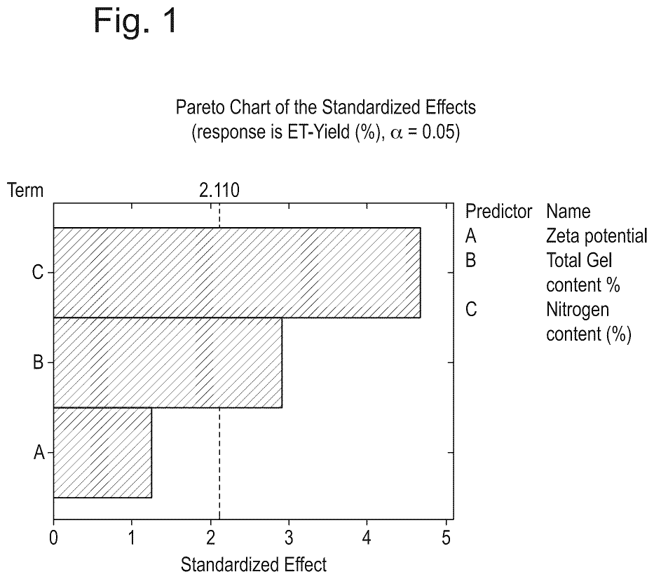

FIG. 1 shows the Pareto Chart for the multiple regression analysis carried out in Example 5. The chart shows the significance of the effect of three CNRL parameters measured in Example 2: zeta potential (A), total gel content (B) and nitrogen content (C) on the electronic testing yield (ET-yield) as measured in Example 4.

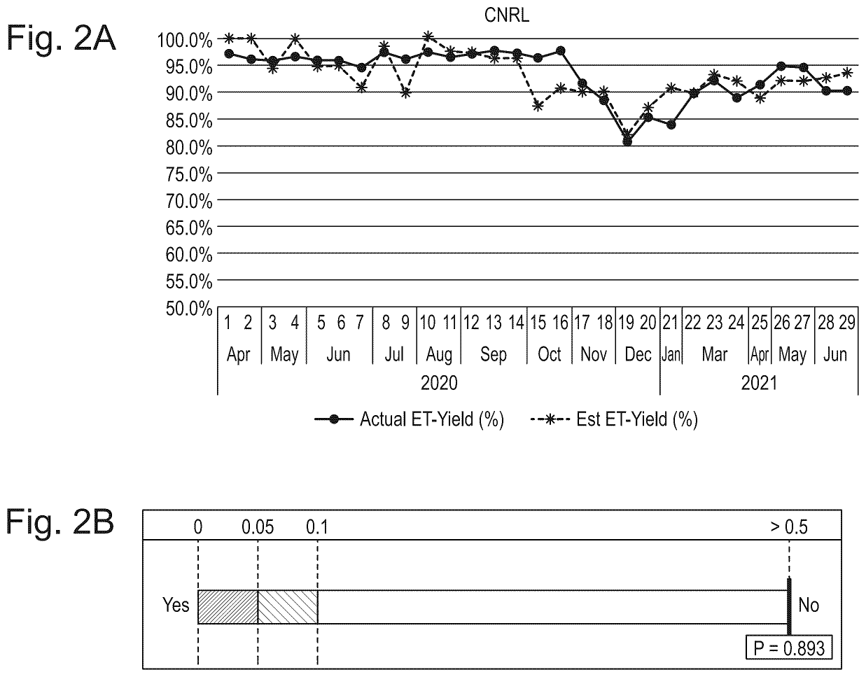

FIG. 2 compares the actual ET-yield with the estimated ET-yield for condoms produced from different CNRL batches from the same supplier from April 2020 to June 2021.

In FIG. 2A, the ET-yield is shown on the y-axis and the month each batch of condoms was tested is shown on the x-axis, with the numbers representing batch numbers. There is a line for the actual ET-yield and the estimated ET-yield.

In FIG. 2B, “Yes” indicates there is a difference between the actual and estimated ET-yields and “No” indicates that there is no difference.

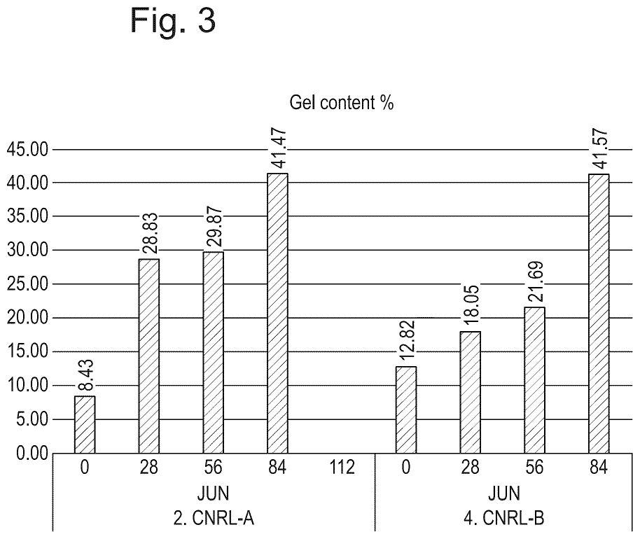

FIG. 3 shows the total gel content for two batches of CNRL (CNRL-A and CNRL-B) and how this varies with storage time. The total gel content (%) is shown on the y-axis and the storage time (days) is shown on the x-axis.

The present invention will now be described in relation to the following non-limiting Examples.

### EXAMPLE 1

The inventors sought to investigate whether there is a correlation between certain properties of concentrated natural rubber latex (CNRL) and the electronic testing percentage yield.

42 different batches of CNRL were obtained from the same commercial supplier from May 2020 until September 2021. For each batch, the following parameters were measured as detailed in Example 2:
Zeta potential
Total gel content
Hard gel content
Nitrogen content

Each individual batch of CNRL was then used to prepare a batch of condoms. For each batch of condoms, the electronic testing percentage yield (ET-yield), was measured as detailed in Example 3.

A correlation analysis was carried out between the ET-yield and each of the above parameters in order to assess the impact of each parameter on condom quality.

### EXAMPLE 2

### Zeta Potential

Each individual batch of CNRL was diluted to 0.05% w/v (0.1 g of NRL in 200 ml) with pH-adjusted deionised water (pH 10.5), which was prepared by using 0.1 M NaOH as pH adjuster. A Malvern Panalytical Zetasizer instrument was used to measure the zeta potential of the resulting latex. For each batch of CNRL, the average zeta potential was calculated from five replicate results. A reflective index of 1.52 and absorbance of 0.001 was used.

### Total Gel and Hard Gel Contents

The total gel content of each individual batch of CNRL was determined by dissolving dry rubber in dry toluene to make a concentration of 1.0% w/v and keeping it in the dark without stirring for one week at room temperature to attain equilibrium. The solution was centrifuged at 1000 rpm for 30 minutes to separate the gel fraction. The gel fraction was coagulated using MeOH and dried in vacuum at 40° C. until a constant weight was obtained. The gel content was calculated as the weight ratio of the gel fraction to the original rubber.

The hard gel content of the gel fraction was measured by analysis of insoluble fraction of rubber after soaking in dried toluene with 1% EtOH. The so-called soft gel is solubilized by addition of the polar solvent EtOH into the rubber solution.

### Nitrogen Content

The Nitrogen content in NR was measured using a Nitrogen Analyzer (Leco instrument, FP-528). About 0.25 g of a rubber sample from each batch of CNRL was accurately measured in an aluminium pan. The nitrogen content was calculated from triplicate analysis, SD≤0.0015%, as comparable to EDTA standard and the calculation was performed using the built-in software.

### EXAMPLE 3

A batch of condoms was prepared from each of the 42 batches of incoming latex that were analysed in Example 2. The dipping machine and the type of condoms was the same for each batch of condoms.

Each individual batch of latex was used to prepare a compounded natural rubber latex comprising the latex and small quantities of stabilisers, vulcanising agents, activators and the like. The same compounding ingredients and amounts were used and the same compounding process was performed in each case.

Condoms were prepared from each batch of compounded natural rubber latex. Again, the same process steps, reagents and conditions were used in each case.

Glass formers were dipped into the compounded natural rubber latex twice to prepare two layers of NRL film on each former, resulting in condoms having a straight wall thickness of approximately 70 μm. After each step of dipping, the films were dried on the formers. Following the second drying step, brushes were used to roll the tops of the condoms to form a bead at the end of each condom whilst the formers rotate to prevent defective beads. The formers then moved into an oven and the films were vulcanised.

Following vulcanisation, the condoms were leached by immersing the formers in an NaOH solution, allowing the condoms to be stripped from each former using a water strip without sticking or creasing. The stripped condoms were then collected and coated with a finishing powder.

Electronic testing (ET) was performed on each finished condom in the batch. The condom was loaded carefully onto a mandrel and a voltage of 1800±100 V was applied, decreasing until any holes were detected. A threshold of 1000 V was defined, meaning that condoms failed the test if a hole or holes were detected at a voltage of greater than 1000 V and passed if a hole or holes were only detected at a voltage of less than 1000 V. The % ET yield of each condom batch was calculated using the following equation:

$$\% ET yield = [ Number of accepted condoms / Total number of condoms ] \times 100$$

### EXAMPLE 4

A bivariate Pearson correlation analysis was carried out to determine the Pearson correlations between CNRL parameters detailed in Example 2 and condom parameters in Example 4. A Pearson correlation value of 0.35-0.65 indicates a moderate correlation, whereas a Pearson correlation value of >0.65 indicates a strong correlation. The results are shown in Table 1 below.

| | ET-Yield | BP | BV |
|---|---|---|---|
| Variables | (%) | (kPa) | (Liter) |
| Zeta potential. | 0.581 | 0.006 | 0.33 |
| Total Gel content | −0.612 | −0.104 | −0.556 |
| % | | | |
| Hard gel (%) | −0.475 | −0.286 | −0.686 |
| Nitrogen content | 0.703 | 0.486 | 0.305 |
| (%) | | | |

It was observed that the electronic testing percentage yield (ET-yield) had a strong positive correlation with the nitrogen content, a moderate negative correlation with the hard gel content and the total gel content and a moderate positive correlation with the zeta potential.

Best Subsets Regression was used to compare different regression models that contain subsets of three CNRL parameters—the zeta potential, the total gel content and the nitrogen content. The results are shown in Table 2.

| | ET-Yield | BP | BV | | ET-Yield |
|---|---|---|---|---|---|
| Variables | (%) | (kPa) | (Liter) | Variables | (%) |
| Zeta | 0.581 | 0.006 | 0.33 | Zeta | 0.581 |
| potential. | | | | potential. | |
| Total Gel | −0.612 | −0.104 | −0.556 | Total Gel | −0.612 |
| content % | | | | content % | |
| Hard gel (%) | −0.475 | −0.286 | −0.686 | Hard gel | −0.475 |
| | | | | (%) | |
| Nitrogen | 0.703 | 0.486 | 0.305 | Nitrogen | 0.703 |
| content (%) | | | | content (%) | |
| Zeta | 0.581 | 0.006 | 0.33 | Zeta | 0.581 |
| potential. | | | | potential. | |
| Total Gel | −0.612 | −0.104 | −0.556 | Total Gel | −0.612 |
| content % | | | | content % | |
| Hard gel (%) | −0.475 | −0.286 | −0.686 | Hard gel | −0.475 |
| | | | | (%) | |

The model with all three has the lowest value of S, the highest value of adjusted R² and smallest value of Mallows' Cₚ.

A multiple regression analysis was carried out to determine the relationship between the zeta potential, the total gel content and the nitrogen content and the ET-yield. The results are shown in Tables 3 and 4.

### Regression Equation

$$ET - Yield ( \% ) = 0.551 + 0.0 \; 0254 [ Zeta potential ] - 0.00296 Total \\ \\ gel content \% + 1.315 Nitrogn content ( \% )$$

### Analysis of Variance

| Source | DF | Adj. SS | Adj. MS | F-value | P-value |
|---|---|---|---|---|---|
| Regression | 3 | 0.031601 | 0.010534 | 23.37 | 0.000 |
| Zeta potential | 1 | 0.000714 | 0.000714 | 1.58 | 0.225 |
| Total gel | 1 | 0.003821 | 0.003821 | 8.48 | 0.010 |
| content | | | | | |
| Nitrogen | 1 | 0.009848 | 0.009848 | 21.85 | 0.000 |
| content | | | | | |
| Error | 17 | 0.007663 | 0.000451 | | |
| Total | 20 | 0.039264 | | | |

### Model Summary

| S | R-sq | R-sq (adj.) | R-sq (pred.) |
|---|---|---|---|
| 0.0212318 | 80.48% | 77.04% | 70.34% |

The multiple regression analysis suggests that there is a positive correlation between (i) the zeta potential and (ii) the nitrogen content with the ET-yield, and a negative correlation between the total gel content and the ET-yield.

The p-values for the total gel content and the nitrogen content are less than the significance level of 0.05, indicating that these parameters both have a significant effect on the ET-Yield.

FIG. 1 shows the Pareto chart of standardised effects from the largest effect to the smallest effect. The bars for the total gel content (B) and the nitrogen content (C) cross the reference line at 2.110, indicating that the effects of the gel content and the nitrogen content on the ET-yield are statistically significant (p<0.05).

This experiment illustrates the impact of the gel content on the ET-yield for condoms having a normal thickness (70 μm). The effect of the gel content is expected to be even more pronounced for thin condoms (for example condoms having a thickness of less than 55 μm) which would be more sensitive to the presence of defects.

The three parameters in the regression equation were measured for batches of concentrated rubber latex obtained from the same commercial supplier from April 2020 to June 2021. The regression equation was used to predict the ET-yield for each batch and the predicted ET-yield was compared with the actual ET-yield measured for condoms prepared from each batch. The comparison of the predicted and actual ET-yields is shown in FIG. 2. The Paired T-Test p value was 0.893, which is higher than the significance level of 0.05, meaning that the difference between the estimated and actual ET-yields were not statistically significant. This suggests that the regression equation is a good predictor for determining the actual ET-yield.

### EXAMPLE 5

The total gel content was measured during storage for two batches of CNRL. The zinc content for each batch of CNRL was measured on day 0 of the storage period. The first batch, CNRL-A, had a high zinc content (465.5 ppm) and the second batch, CNRL-B, had a low zinc content (32.4 ppm). Each batch of CNRL was stored in a storage vessel at room temperature and the total gel content was measured on days 0, 28, 56 and 84.

The total gel content of both batches of CNRL increased during the storage period. However, after 56 days of storage, the low-zinc batch had a lower gel content compared to the high-zinc batch. This suggests that the formation of a zinc-amine complex contributes to the increase in gel content with storage time.

Further experiments were run to determine the appropriate zinc content and it was discovered that <60 ppm was the level found to limit the gel formation to an adequate degree.

The foregoing detailed description has been provided by way of explanation and illustration, and is not intended to limit the scope of the appended claims. Many variations in the presently preferred embodiments illustrated herein will be apparent to one of ordinary skill in the art, and remain within the scope of the appended claims and their equivalents.

1. A method for preparing a plurality of condoms, comprising:
(i) compounding one or more compositions comprising natural rubber latex to make a first batch of compounded latex;
(ii) compounding one or more compositions comprising natural rubber latex to make a second batch of compounded latex;
(iii) optionally, blending said first and second batches of compounded latex to make a compounded latex blend; and
(iv) dipping a plurality of formers into the compounded latex blend to form a plurality of condoms, or
(v) dipping a plurality of formers into the first batch of compounded latex to form a plurality of condoms and then dipping said plurality of formers into the second batch of compounded latex to form a plurality of condoms;
wherein each of the one or more compositions comprising natural rubber latex, which are used to make the first and second batch of compounded latex, has a zinc content less than 60 ppm.

2. The method according to claim 1, wherein each of the one or more compositions comprising natural rubber latex has a zinc content less than 50 ppm.

3. The method according to claim 1, wherein each of the one or more compositions comprising natural rubber latex is preserved with a preservative comprising zinc before compounding.

4. The method according to claim 1, wherein each of the one or more compositions comprising natural rubber latex has an ammonia content of from 0.65 to 1.5 wt %.

5. The method according to claim 1, wherein each of the one or more compositions comprising natural rubber latex is stored for a storage period of at least 30 days before compounding.

6. The method according to claim 5, wherein the zinc content of each of the one or more compositions comprising natural rubber latex is maintained at a level less than 60 ppm throughout said storage period.

7. The method according to claim 1, wherein each of the plurality of condoms has a thickness of from 35 to 55 μm.

8. The method according to claim 1, further comprising electronic testing of each of the plurality of condoms, wherein at least 90% of the condoms of the plurality of condoms obtained by the method pass the electronic testing and all those condoms which do not pass the electronic testing are discarded.

9. A plurality of condoms obtained by the method of claim 1.

10. The method according to claim 2, wherein each of the one or more compositions comprising natural rubber latex has a zinc content less than 30 ppm.

11. The method according to claim 10, wherein each of the one or more compositions comprising natural rubber latex has a zinc content less than 20 ppm.

12. The method according to claim 5, wherein each of the one or more compositions comprising natural rubber latex is stored for a storage period of at least 42 days before compounding.
